(19) United States
(12) Patent Application Publication
(10) Pub. No.: US 2024/0392246 A1
(43) Pub. Date: Nov. 28, 2024

(54) Method for the Generation of Non-Transformed Macrophage Cell Line

(71) Applicant: UNIVERSITY OF PLYMOUTH, Plymouth (GB)

(72) Inventors: Gyorgy Fejer, Plymouth (GB); Justyna Lopatecka, Plymouth (GB)

(21) Appl. No.: 18/695,770

(22) PCT Filed: Sep. 22, 2022
(86) PCT No.: PCT/EP2022/076456

§ 371 Date: Mar. 26, 2024

(30) Foreign Application Priority Data
Sep. 27, 2021 (GB) 2113747.6
Jul. 9, 2022 (GB) 2210102.6
Mar. 22, 2024 (GB) 2404177.4

(51) Int. Cl. C12N 5/0786

(52) U.S. Cl. CPC C12N 5/0645; C12N 2501/052; C12N 2501/22; C12N 2502/27

(57) ABSTRACT

A method for producing continuously replicating, non-transformed pig macrophages, comprising culturing a cell preparation from an organ obtained from a pig in culture medium to which GM-CSF has been added thereby differentiating the cell population into self-renewing, non-transformed macrophages.

The present invention relates generally to macrophages and particularly, although not exclusively, to the establishment of GM-CSF dependent, self-renewing, non-transformed pig macrophages.

### INTRODUCTION

Macrophages are specialised cells involved in the detection, phagocytosis and destruction of bacteria and other harmful organisms. In addition, they can also present antigens to T cells and initiate inflammation by releasing molecules (known as cytokines) that activate other cells.

Macrophages are crucial in defence against infection and studies with these cells in vitro are key to understanding host-pathogen interactions and to underpin vaccine development.

Depending on their development and organ distribution, macrophages have very distinct properties. Previously, all tissue resident macrophages, including alveolar macrophages (AMs) were believed to be bone-marrow hematopoietic stem cell (HSC) derived cells with a limited life-span. Recent studies however, demonstrated that most tissue resident macrophages represent a separate, functionally distinct, embryonic macrophage-derived lineage (1). Most tissue-resident macrophages are macrophage colony-stimulating factor (M-CSF) driven self-renewing cells, while AMs are granulocyte-macrophage colony-stimulating factor (GM-CSF) dependent, autonomously growing cells. AMs have unique characteristics among macrophages including sensitivity and responses to pathogens (2).

Macrophages are best investigated using primary cells because transformed cell lines may not accurately represent the primary cells. Macrophage pathogenesis studies mostly use cells isolated directly from organs or use ex vivo produced M-CSF derived macrophages from bone marrow progenitors or peripheral blood mononuclear cells (PBMC) (3). GM-CSF derived macrophages from human PBMCs can be used to model human lung AMs (4). Nevertheless, these methods provide cells with a limited life-span, restricted availability, are difficult to genetically manipulate and significant inter-donor variability limits their use.

Mouse models including macrophage systems are often used to study human and veterinary infections because of the relatively easy access to tissues and experiments in vivo. However, there are significant differences regarding macrophage responses and/or intracellular pathogen replication between different mammalian species, which limits the utility of murine studies (5).

Recently we established a novel, non-transformed, continuously growing, murine macrophage model (MPI cells) from foetal liver providing unlimited amounts of primary cells. This robust system faithfully reproduces murine AM specific responses to various respiratory pathogens in vitro (6).

Macrophage studies in pigs are very important (5). Pigs are highly susceptible to various viral (e.g. African swine fever virus [ASFV], porcine reproductive and respiratory syndrome virus [PRRSV] and influenza A virus [IAV]) and bacterial (e.g. Mycobacterium avium, and Salmonella spp.) pathogens that replicate within macrophages and/or profoundly alter macrophage functions (7-9). Some of these pathogens spread from pigs to humans and cause serious diseases in them (e.g. IAV) (9). Furthermore, swine models are used increasingly in biomedical research as pig and human physiology, such as the anatomy and function of the airways and disease susceptibility are much closer to each other than to those of mice (10).

M-CSF can be used to differentiate pig macrophages from monocytes and detailed analysis of LPS stimulated macrophages showed a close functional relationship between pig and human MCSF derived macrophages (5). However, these MCSF derived macrophages need to be differentiated freshly from bone marrow and similarly to mouse and human cells they have a limited life-span, which makes them unsuitable to produce viral vaccines and suitable, robust primary, non-transformed continuous pig macrophage cell lines are not available for research and vaccine production.

Such macrophage lines would be required to study important pathogens such as IAV, ASFV and PRRSV.

Humans and pigs are natural hosts for IAV and the disease can spread in both directions between these species (9). IAV is the leading infectious cause of human death. AMs and innate immune responses play key roles in the pathogenesis of IAV infection (11). Pigs are susceptible to a wide range of IAV strains and are considered to be the “mixing vessel” of these pathogens as both avian and human IAV strains replicate in pigs and coinfection may lead to new viruses such as the triple (bird-human-pig) reassortant H1N1 strain responsible for the most recent pandemic in 2009.

ASFV and PRRSV are pathogens of huge economic importance. These viruses replicate almost exclusively in macrophages, thus, for their efficient study and vaccine development establishment of a continuously growing permissive macrophage system would be necessary (7, 8). ASFV causes a devastating, Ebola disease-like haemorrhagic syndrome in pigs. It kills almost all infected animals and in 2018 and 2019 resulted in the death of almost 1.5 million pigs and 10,000 wild boar. The lack of a vaccine limits disease control and research is directed to better understand how the virus interacts with and modulates macrophage functions. This is important, as potential vaccine strains deleted of anti-interferon viral genes elicit a vigorous innate response that restrict viral growth and prevent efficient vaccine production in normal macrophages (7).

PRRSV is one of the most economically significant swine pathogens, endemic in the majority of pork producing countries, and lung AMs are primary targets of the virus in natural infections (8). There are two distinct PRRSV species which are rapidly diversifying and the emerging highly pathogenic strains spread with high speed and cause devastating effects. Vaccination is key to control the disease, however, available vaccines are not sufficiently protective and there is a great need to develop safer and more immunogenic vaccines to which a suitable in vitro system is crucially important.

The present invention relates to the establishment of a GM-CSF derived, continuous, non-transformed pig macrophage system.

A method for producing continuously replicating, non-transformed porcine macrophages, comprising culturing a cell preparation from an organ obtained from a pig in culture medium to which GM-CSF has been added thereby differentiating the cell population into self-renewing, non-transformed macrophages.

An aspect of the present invention provides a method for producing continuously replicating, non-transformed pig macrophages, comprising culturing a cell preparation from an organ obtained from a pig in culture medium to which GM-CSF has been added thereby differentiating the cell population into self-renewing, non-transformed macrophages.

The cell preparation may be cultured with feeder cells such as mesenchymal feeder cells.

Macrophages prepared using the method may be characterised by the expression of pig macrophage markers CD163 and CD172a.

The cell preparation may be based on pig foetal spleen cells.

The present invention also provides macrophages obtained or obtainable by the method as described and/or defined herein.

The present invention also provides macrophages obtainable by the method described herein for use in medicine and/or medical/pharmaceutical research.

The present invention also provides macrophages obtainable by the method described herein for use in the production of a vaccine.

The present invention also provides a GM-CSF derived, continuously replicating, non-transformed pig macrophage cell line.

The macrophages may be characterised by the expression of pig macrophage markers CD163 and CD172a.

The present invention also provides a continuously replicating, non-transformed pig macrophage system.

The system may be characterised by the expression of pig macrophage markers CD163 and CD172a.

Some aspects and embodiments of the present invention are based on the principle or observation that continuously replicating non-transformed macrophages can be obtained from pig haematopoetic organs by using GM-CSF and suitable mesenchymal feeder cells.

The following data has been obtained related to this system:
1. We cultured pig foetal spleen cells with 20 μg/ml GM-CSF in order to differentiate macrophages. After regular media changes and replenishment of GM-CSF cultures containing two different cell types developed. Flat, mesenchymal or fibroblast like cells on which round, loosely attached macrophage like cells grew in clumps and some of these latter cells also floated in the medium. GM-CSF was required to the development of both cells as spleen cell cultures without it did not produce viable cells. These cultures could be maintained for at least a year with regular media changes.
2. The floating macrophage-like cells were tried to culture separately with GM-CSF. These cells while stayed alive for weeks did not proliferate further. Separated floating cells were also cultured with MCSF or GM-CSF and MCSF. These MCSF cultures showed initial robust cell proliferation; however, after a week of growth cells stop to multiply.
3. The floating macrophage-like cells have been transferred to embryonic pig lung fibroblast cultures and similarly to the original cultures with the spleen derived fibroblasts, they attached and proliferated on the top the lung fibroblast feeder cells. They grew in clumps and many of these cells were floating in the medium. These cultures were maintained for more than year with regular media changes.
4. The floating cells from the lung fibroblast feeder culture have been cultured with GM-CSF but without feeder cells. These cells survive for extended time intervals but don't grow further significantly.
5. Isolated floating cells from feeder containing cultures strongly express the pig macrophage markers CD163 and CD172a.
6. Isolated floating macrophages from feeder containing cultures produce a strong cytokine response to bacterial endotoxin (LPS) stimulation.
7. Isolated floating macrophages from feeder containing cultures can be serially passaged on Mitomycin treated STO fibroblast line feeder cells with pig GM-CSF for at least 4 passages. They grow continuously and have a doubling time of approximately 3-4 days.
8. When culturing pig foetal bone marrow with GM-CSF, a culture similar to the one from spleen develops with mesenchymal/fibroblast like adherent cells and floating macrophage like cells. The floating cells proliferate in the presence of GM-CSF when transferred to mytomycin treated STO fibroblast cells, however, more slowly than similar cells from the spleen.
9. Isolated floating macrophages from the feeder cultures have been transduced with a 2^{nd} generation lentivirus expressing the SV40 Large T antigen tsA58 temperature sensitive mutant (Jat, P. S. & Sharp, P. A. (1989) Mol. Cell. Biol. 9, 1672-1681.). The tsA58 mutant of the SV40 Large T antigen is thermosensitive and can be used to generate conditionally immortalized cell lines (Jat, P. S. & Sharp, P. A. (1989) Mol. Cell. Biol. 9, 1672-1681.) The obtained cells (known as PLTA58 cells) grow robustly and without the need of feeder cells. These data represent a significant difference to previous data by Takenouchi et al, Front Vet Sci 2017 Aug. 21;4:132, where unconditional cellular immortalisation of pig kidney derived macrophages could only be achieved by the combined expression of the telomerase protein and the wild type SV40 large T antigen.
10. PLTA58 cells are continuously growing without feeder cells and we have carried out at least 20 passages since their establishment in December 2021. We can regularly obtain at least 10 million cells from a T75 culture flask in one week of culture. We can freeze down the cells and culture them efficiently again after thawing.
11. PLTA58 cells are factor dependent and their growth is enhanced by GMCSF and/or MCSF.
12. PLTA58 cells express pig macrophage markers.
13. PLTA58 cells can be efficiently stimulated with various innate response elicited ligands such as LPS (TLR4 ligand), Fsl-1 (TLR2 ligand), polyl:C (TLR3 ligand) and R848 (TLR7/8 ligand).
14. PLTA58 cells support the replication of the African Swine Fever Virus.

Different aspects and embodiments of the invention may be used separately or together.

Further particular and preferred aspects of the present invention are set out in the accompanying independent and dependent claims. Features of the dependent claims may be combined with the features of the independent claims as appropriate, and in combinations other than those explicitly set out in the claims.

The present invention is more particularly shown and described, by way of example, in the accompanying drawings.

The example embodiments are described in sufficient detail to enable those of ordinary skill in the art to embody and implement the systems and processes herein described. It is important to understand that embodiments can be provided in many alternative forms and should not be construed as limited to the examples set forth herein.

Accordingly, while embodiments can be modified in various ways and take on various alternative forms, specific embodiments thereof are shown in the drawings and described in detail below as examples. There is no intent to limit to the particular forms disclosed. On the contrary, all modifications, equivalents, and alternatives falling within the scope of the appended claims should be included.

Unless otherwise defined, all terms (including technical and scientific terms) used herein are to be interpreted as is customary in the art. It will be further understood that terms in common usage should also be interpreted as is customary in the relevant art and not in an idealised or overly formal sense unless expressly so defined herein.

In the description, all orientational terms, such as upper, lower, radially and axially, are used in relation to the drawings and should not be interpreted as limiting on the invention.

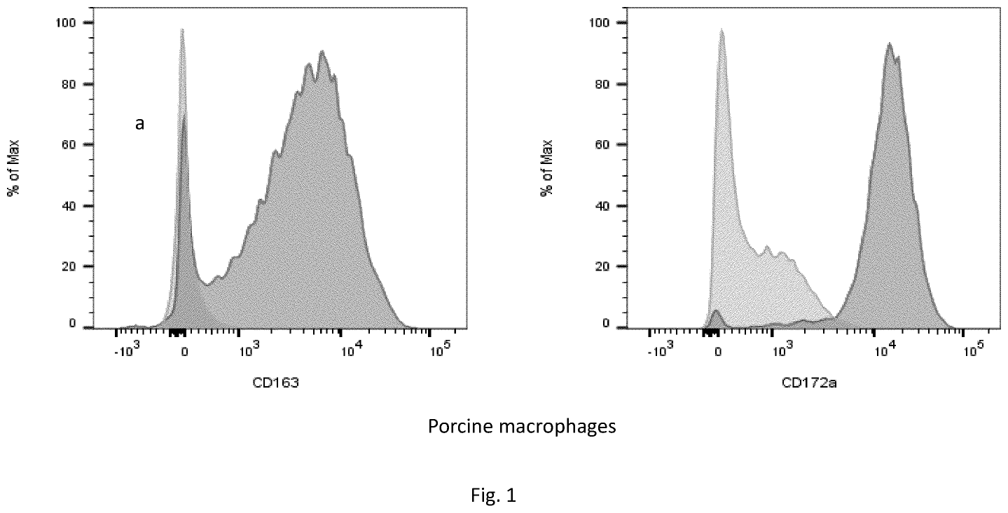

FIG. 1

Continuously growing porcine GMCSF derived cells express markers characteristic for pig macrophages.

Surface markers were detected on porcine macrophages with FACS using the antibodies for the scavenger receptor CD163 and signal regulatory protein alpha CD172a. Histograms show unstained (blue) and stained cells (red).

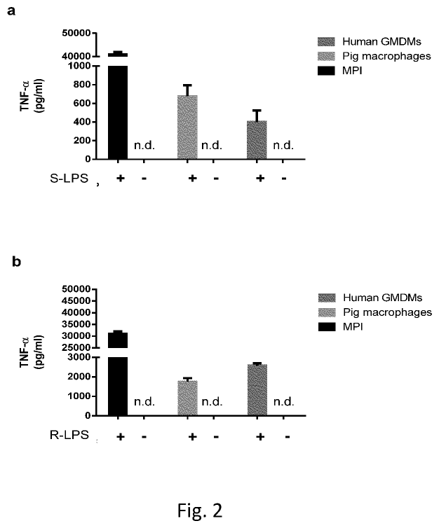

FIG. 2

In response to smooth and rough form LPS the levels of pig macrophage produced TNFa is similar to those of human macrophages.

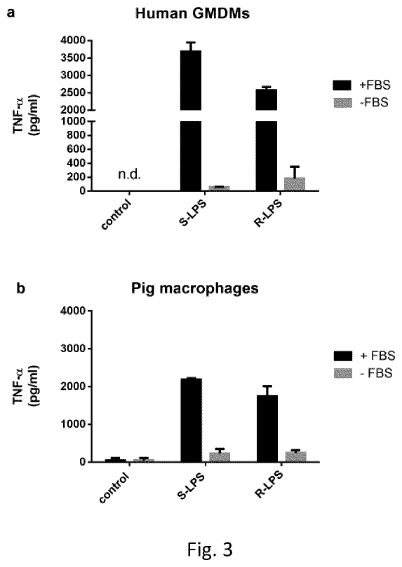

FIG. 3

TNF-α response in human GMDMs and pig macrophages is highly dependant on LBP present in Foetal Bovine Serum (FBS). Cells were stimulated with S-LPS (100 ng/ml) and R-LPS (100 ng/ml). At 16 h post-infection supernatants were collected and TNF-α was measured by ELISA. Bars represent the mean from three sample±S.E.M.

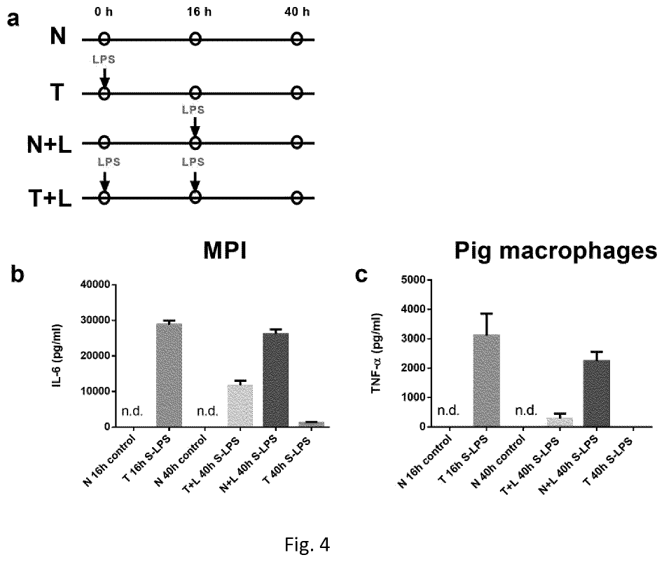

FIG. 4

Reduced production of IL-6 and TNF-α in MPI and pig macrophages respectively to repeated LPS stimulations (LPS tolerance). (a) cells were unstimulated (N) or stimulated with 50 ng/ml S-LPS for 16 h (T), washed with PBS and replenished with media (N) or 50 ng/ml LPS (N+L or T+L) for 24 h. Arrows depict stimulation with LPS. Supernatants from MPI (b) and pig macrophages (c) were analysed by ELISA.

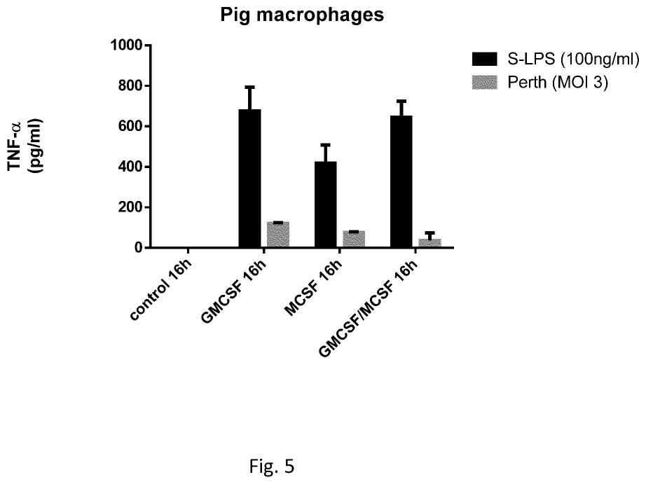

FIG. 5

Comparable TNF-α cytokine production upon S-LPS and IAV in pig macrophages cultured under various conditions. Pig cells were stimulated with Influenza A virus strain Perth/16/09 at MOI 3 and S-LPS (100 ng/ml). At 16 h post-infection supernatants were collected and TNF-α were measured by ELISA. n=1

Similarities between the responses of M-CSF, GM-CSF and GM-CSF/M-CSF-cultured porcine macrophages after obtaining them from lung fibroblast feeder cultures with GM-CSF.

The first set of questions involving a newly developed model of pig macrophages aimed to compare those cells growing in different conditions. The data demonstrate that these pig macrophages can be efficiently stimulated when cultured without feeder cells with the growth factors used.

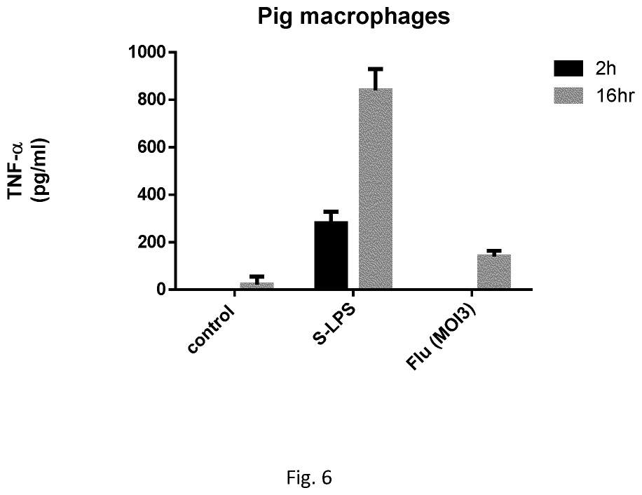

FIG. 6

Pig macrophages produce an early TNF-α response after challenge with lipopolysaccharide. Pig cells were stimulated with IAV strain Perth/16/09 at MOI 3 and S-LPS (100 ng/ml). At 16 h post-infection supernatants were collected and TNF-α were measured by ELISA. n=1

TNF-α cytokine production in pig GM-CSF-derived macrophages stimulated with S-LPS and IAV is time-dependent.

The next experiment was concerned about significant proinflammatory cytokine production in porcine macrophages challenged with S-LPS and IAV at various time points. FIG. 6 shows early TNF-α production in pig macrophages stimulated with S-LPS but not IAV. Additionally, the IAV responses are lower compared to those elicited by endotoxin.

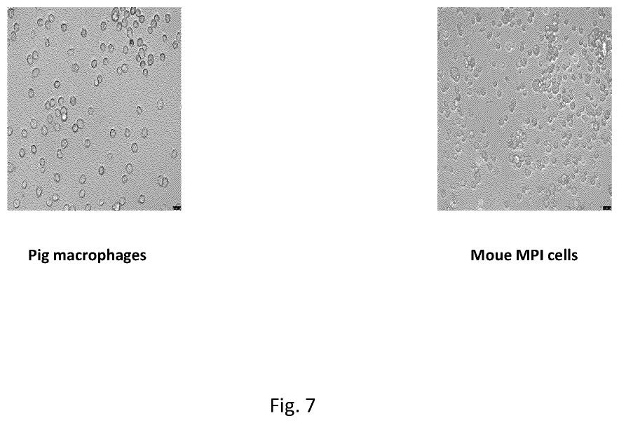

FIG. 7

Pig and MPI macrophages transduced with RFP expressing lentivirus. These data demonstrate that the pig macrophages can be efficiently targeted for recombinant protein expression by lentiviral vectors similarly to mouse MPI macrophages.

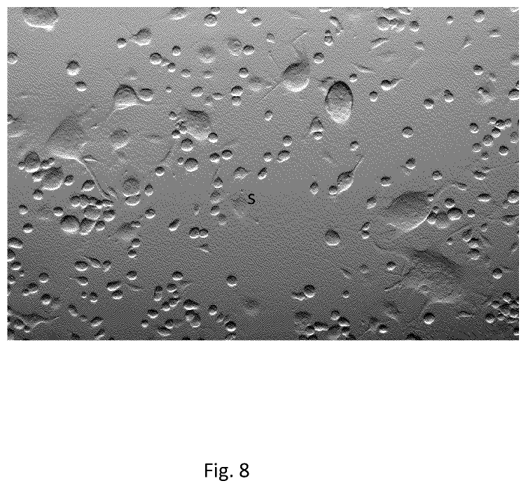

FIG. 8

Extended culture of pig macrophages with GM-CSF separated from feeder cells. Multinuclear giant cells, characteristic for macrophages can be seen among regular sized cells.

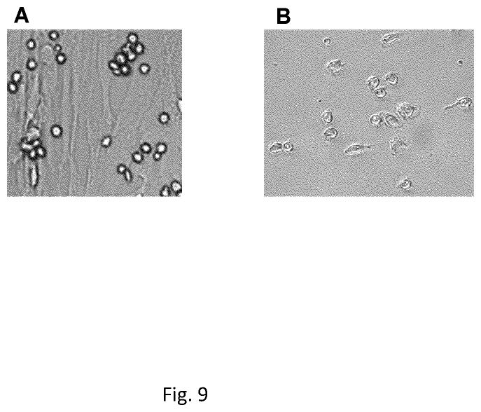

FIG. 9

Pig macrophage cells growing on lung fibroblast feeder cells (A) and transferred separated floating macrophages without feeder (B).

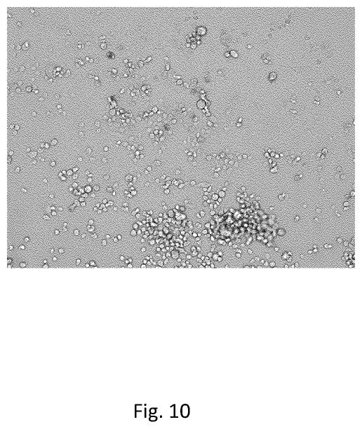

FIG. 10

Pig macrophages grown on the STO fibroblast cell line.

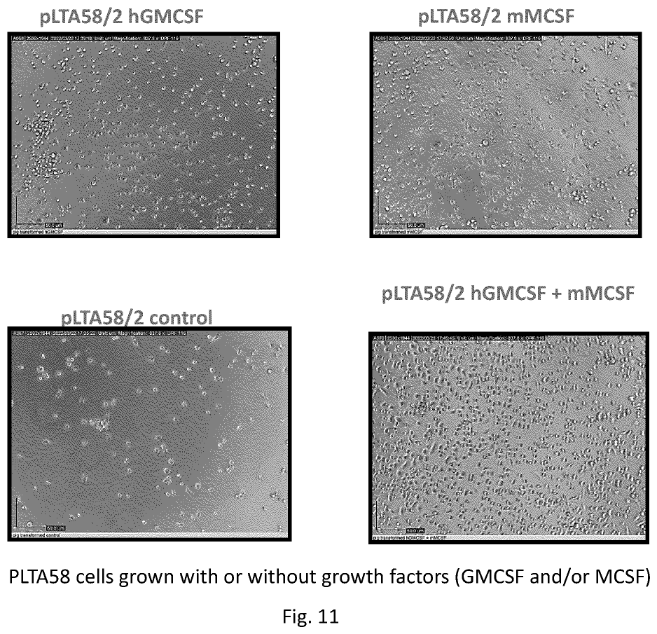

FIG. 11

Factor dependency of PLTA58 cells shown by cultures with or without growth factors (GMCSF and/or MCSF).

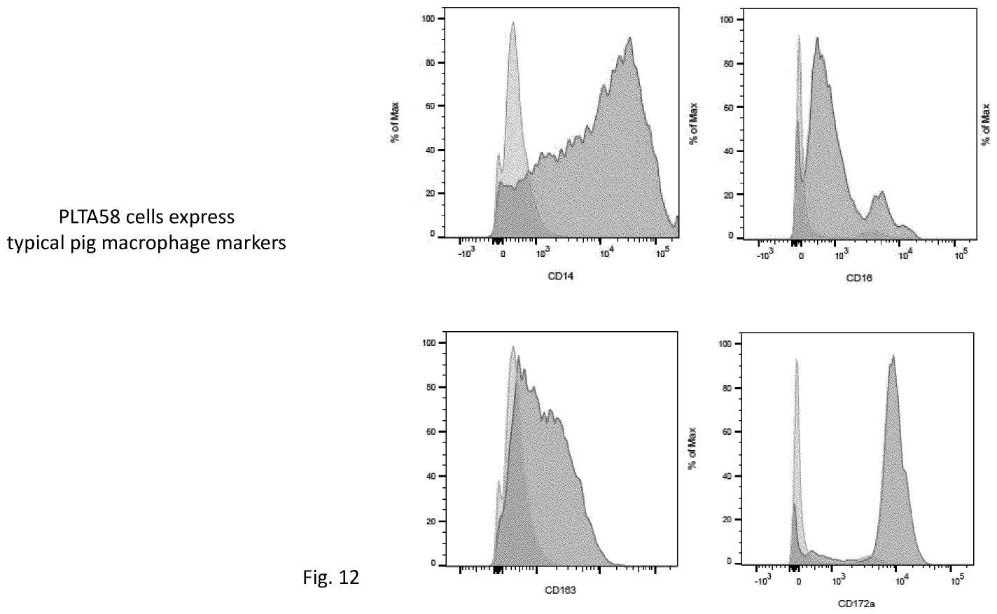

FIG. 12

FACS data on the immortalized pig cells.

PLTA58 cells express typical pig macrophage markers.

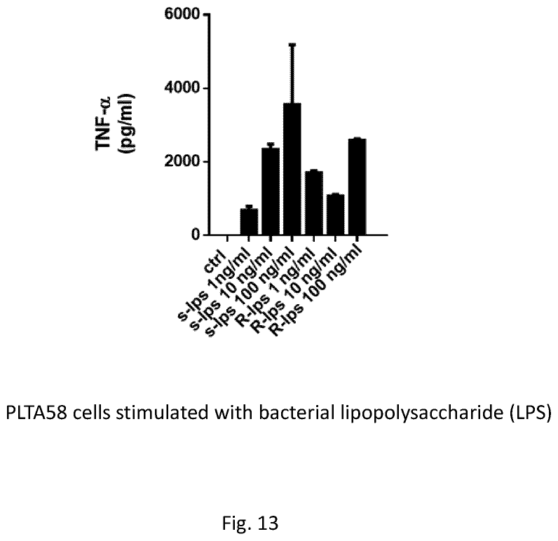

FIG. 13

PLTA58 cells stimulated with bacterial lipopolysaccharide (LPS)

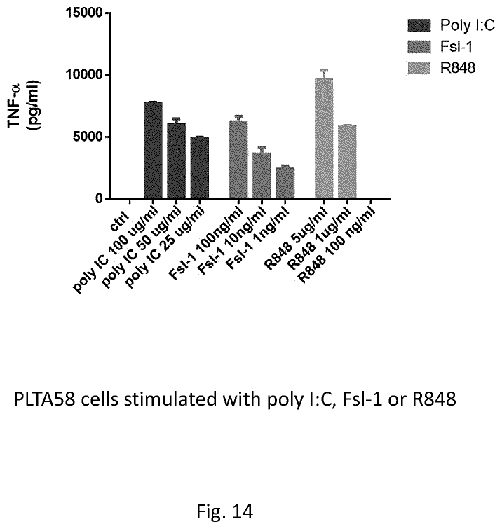

FIG. 14

PLTA58 cells stimulated with poly I:C, Fsl-1 or R848.

Although illustrative embodiments of the invention have been disclosed in detail herein, with reference to the accompanying drawings, it is understood that the invention is not limited to the precise embodiments shown and that various changes and modifications can be effected therein by one skilled in the art without departing from the scope of the invention as defined by the appended claims and their equivalents.

### REFERENCES

1. Guilliams M, Mildner A, & Yona S (2018) Developmental and functional heterogeneity of monocytes. Immunity 49(4):595-613.
2. Hussell T & Bell T J (2014) Alveolar macrophages: plasticity in a tissue-specific context. Nature reviews immunology 14(2):81-93.
3. Geissmann F, Gordon S, Hume D A, Mowat A M, & Randolph G J (2010) Unravelling mononuclear phagocyte heterogeneity. Nature Reviews Immunology 10(6):453.
4. Akagawa K S, et al. (2006) Functional heterogeneity of colony-stimulating factor-induced human monocyte-derived macrophages. Respirology 11:S32-S36.
5. Kapetanovic R, et al. (2012) Pig bone marrow-derived macrophages resemble human macrophages in their response to bacterial lipopolysaccharide. The Journal of Immunology 188(7):3382-3394.
6. Fejer G, et al. (2013) Nontransformed, GM-CSF-dependent macrophage lines are a unique model to study tissue macrophage functions. Proceedings of the National Academy of Sciences 110(24):E2191-E2198.
7. Dixon L, Islam M, Nash R, & Reis A (2019) African swine fever virus evasion of host defences. Virus research.
8. Singleton H, Graham S P, Bodman-Smith K B, Frossard J-P, & Steinbach F (2016) Establishing porcine monocyte-derived macrophage and dendritic cell systems for studying the interaction with PRRSV-1. Frontiers in microbiology 7:832.
9. Nelson M I & Vincent A L (2015) Reverse zoonosis of influenza to swine: new perspectives on the human-animal interface. Trends in microbiology 23(3):142-153.
10. Walters E M, Wells K D, Bryda E C, Schommer S, & Prather R S (2017) Swine models, genomic tools and services to enhance our understanding of human health and diseases. Lab animal 46(4):167.
11. Pulendran B & Maddur M S (2015) Innate Immune Sensing and Response to Influenza. Current topics in microbiology and immunology 386:23.
12. Lo lacono M, et al. (2018) Wharton's Jelly Mesenchymal Stromal Cells Support the Expansion of Cord Blood-derived CD34+Cells Mimicking a Hematopoietic Niche in a Direct Cell-cell Contact Culture System. (SAGE Publications Sage CA: Los Angeles, CA).

1. A method for producing continuously replicating, non-transformed pig macrophages, comprising culturing a cell preparation from an organ obtained from a pig in culture medium to which GM-CSF has been added thereby differentiating the cell population into self-renewing, non-transformed macrophages.

2. The method according to claim 1, in which the cell preparation is cultured with feeder cells.

3. The method according to claim 2, in which the feeder cells comprise mesenchymal feeder cells.

4. The method according to claim 1, wherein macrophages are characterised by the expression of pig macrophage markers CD163 and CD172a.

5. The method according to claim 1, wherein macrophages are characterised by the expression of Integrin alpha M (CD11b) and/or MHC class II cell surface receptor (HLA-DR).

6. The method according to claim 1 based on pig foetal spleen cells.

7. (canceled)

8. The cell line according to claim 10 for use in medicine and/or medical/pharmaceutical research.

9. The cell line according to claim 10 for use in the production of a vaccine.

10. A GM-CSF derived, continuously replicating, non-transformed pig macrophage cell line formed according to the method of claim 1 and in which macrophages are characterised by the expression of pig macrophage markers CD163 and CD172a.

11. (canceled)

12. (canceled)

13. (canceled)

14. The method according to claim 1, further comprising the step of selecting floating macrophages and transferring them to a feeder cell culture.

15. The method according to claim 14, further comprising the step of transducing the floating macrophages from the feeder culture with tsA58 to generate a conditionally immortalized cell line.

16. (canceled)

17. (canceled)

18. (canceled)

19. The cell line according to claim 10 and capable of supporting replication of the African Swine Fever Virus.

20. (canceled)

21. (canceled)

22. (canceled)

23. A method according to claim 1, comprising the steps of:
i) culturing pig foctal spleen cells with GM-CSF in order to differentiate macrophages;
ii) isolating floating macrophages from the culture of step i) and transferring them to a feeder culture of pig feeder cells;
iii) isolating floating macrophages from the feeder culture of step ii), said isolated floating macrophages strongly expressing the pig macrophage markers CD163 and CD172a; and
iv) transducing isolated floating macrophages from step iii) with tsA58 to generate a conditionally immortalized cell line designated PLTA58 which grow robustly and without the need of feeder cells.

24. An isolated cell obtainable from the cell line having ECACC Accession No. 24032101.

25. A cell line deposited under ECACC Accession No. 24032101.

26. The cell or cell line of claim 24 for use in medicine and/or medical/pharmaceutical research.

27. The cell or cell line of claim 24 for use in the production of a vaccine.

28. The cell or cell line of claim 24 and being capable of supporting replication of the African Swine Fever Virus.
